(19) United States
(12) Patent Application Publication
(10) Pub. No.: US 2019/0262609 A1
(43) Pub. Date: Aug. 29, 2019

(54) SPINAL CORD STIMULATION BASED ON PATIENT-SPECIFIC MODELING

(71) Applicant: Boston Scientific Neuromodulation Corporation, Valencia, CA (US)

(72) Inventors: Natalie A. Brill, Valencia, CA (US); Bradley Lawrence Hershey, Valencia, CA (US); Ross D. Venook, Millbrae, CA (US)

(21) Appl. No.: 16/285,799

(22) Filed: Feb. 26, 2019

Related U.S. Application Data
(60) Provisional application No. 62/636,413, filed on Feb. 28, 2018.

(51) Int. Cl. A61N 1/36; A61N 1/05; A61N 1/372; G16H 10/60

(52) U.S. Cl. CPC A61N 1/36062; A61N 1/36071; A61N 1/0551; G16H 50/50; G16H 10/60; A61N 1/37252; A61N 1/37247

(57) ABSTRACT

Systems and methods for controlling delivery of spinal cord stimulation based on a patient-specific computational spinal cord (CSC) model are discussed. An embodiment of a system comprises a data processor to receive a patient dataset representing a neural structure of at least a portion of the patient spinal cord, and extract a feature from the received patient dataset. The system includes a stimulation control circuit to receive a generic CSC model generalized from a patient population that characterizes spinal cord anatomy and physical properties. The stimulation control circuit can generate a patient-specific model by modifying the generic CSC model using the extracted feature, and compute a stimulation parameter value using the patient-specific model. An ambulatory electrostimulator can generate spinal cord stimulation according to the computed stimulation parameter value.

### CLAIM OF PRIORITY

This application claims the benefit of priority under 35 U.S.C. § 119(e) of U.S. Provisional Patent Application Ser. No. 62/636,413, filed on Feb. 28, 2018, which is herein incorporated by reference in its entirety.

### TECHNICAL FIELD

This document relates generally to medical devices and more particularly to systems and methods for controlling spinal cord stimulation using a patient-specific computational model.

### BACKGROUND

Neurostimulation, also referred to as neuromodulation, has been proposed as a therapy for many conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been used clinically to deliver such a therapy. An implantable neurostimulation system typically includes an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system.

The neurostimulation energy can be delivered in the form of electrical stimulation pulses. The delivery is controlled using stimulation parameters that specify spatial (where to stimulate), temporal (when to stimulate), and informational (patterns of pulses directing the nervous system to respond as desired) aspects of a pattern of stimulation pulses. In an example, an electrode combination is used to deliver neurostimulation energy to a targeted tissue, with the electrodes selectively programmed to act as anodes (positive), cathodes (negative), or left off (zero).

To achieve an effective therapeutic outcome from neurostimulation, leads and electrodes thereon need to be placed in appropriate locations of the patient. For example, in SCS for pain management, an array of electrodes may be placed at such body locations that may cause paresthesia, an alternative sensation that replaces the pain signal that is sensed by a patient. The paresthesia induced by electrostimulation and perceived by the patient may be located in approximately the same body locations as the pain that is the target of treatment. Thus, lead electrode positions can be important to achieve effective pain therapy.

A user (e.g., a clinician) may use an external programming device to program stimulation parameters into the implantable neurostimulator. The stimulation parameters characterize, among other things, stimulation intensity, stimulation mode, and manner of neurostimulation energy delivery to the target tissue. In an example, electrostimulation energy may be distributed across an array of electrodes via different electrode configurations. The electrodes may provide different relative percentages of positive and negative current or voltage, which is also known as fractionalized electrode configurations. The increasing number of electrostimulation electrodes available, combined with the ability to generate a variety of complex stimulation pulses, presents a wide selection of stimulation parameters to the clinician or patient.

### SUMMARY

A computerized neurostimulation system may be used to facilitate selection or determination of stimulation parameters to achieve desirable therapeutic outcome, such as SCS to maximize pain relief. The computerized system may be a self-contained hardware/software system, or may be defined predominantly by software running on a computer. In one example, the computerized system may steer stimulation energy (e.g., current or voltage) in accordance with a pre-determined navigation table that defines a series of electrode combinations associated with fractionalized electrode configurations. A user may gradually steer current or voltage from one basic electrode configuration to another, thereby electrically altering the stimulation region along the stimulation leads. While the navigation table is a useful tool for steering current, a limited number of fractionalized electrode configurations can be stored in the navigation table due to memory and time constraints in a SCS system. Additionally, the navigation table is typically lead-type dependent. Because the number, location, or orientation of electrodes may vary from one type of lead to another type, the association between the electrode combinations and fractionalization usually needs to correspond to electrode arrangement on a lead type. Substantial amount of time and effort must be spent in developing navigation tables for every new lead type.

As an alternative to navigation tables, a computational model (e.g., a software package) may be used to automatically determine ideal stimulation parameters to achieve desired therapeutic outcome. The computational model is a valuable tool to understand mechanisms of nerve fiber excitation in an electric field, and the impact of stimulation parameters on nerve excitation. A generic computational model may comprise a mathematical (numerical or analytical) representation of structural and physical properties of a neural target, such as various neuronal structures of a spinal cord. The model is generic in a sense that it may be generalized from a patient population. In an example, the model may include a nonlinear double-cable axon model to predict nerve excitation for different myelinated fiber sizes. The computational model may additionally include a software implementation of a generic current-to-contact mapping algorithm. Such an algorithm accepts relative electrode positions and a representation of a target electric field, and maps the target electric field to polarities and percentages of energy (e.g., current or voltage) associated with an array of electrodes. In contrast to the navigation tables that are specific to a lead design and to electrode location information, the computational model generally calls for the input of electrode locations and information about the desired electric field independently, such that the lead design and update of the computational model do not have to be as tightly connected. The stimulation parameters generated by the computational model may be programmed into an implantable neurostimulator to deliver electrostimulation (e.g., SCS).

As discussed above, the generic computational model is typically generalized across a population. However, inter-subject structural or anatomical differences may exist among patients. For example, location, morphology, or trajectory of a spinal structure (e.g., a dorsal root or a dorsal root ganglion) may vary from one patient to another. On the other hand, the medical condition or functional state of a patient may change over time. Without taking into account such inter-subject differences or intra-subject variation over time, a generic computational model may not always provide ideal stimulation parameters. Therefore, there is a need to improve the model-guided electrostimulation, such as SCS for pain management. The present inventors have recognized that a patient-specific computational model, constructed from a generic computational model, may be used to provide individualized current steering and patient-specific stimulation parameter optimization, thereby improving efficacy of certain neurostimulation therapies and reducing their side effects. Additionally, the patient-specific computational model as discussed in this document may also improve the functionality of a neurostimulation system. For example, the individualized current steering may reduce battery consumption and enhance longevity of an implantable neurostimulator. With improved individualized neurostimulation, patient quality of life can be improved, hospitalization and medical intervention can be reduced, and an overall system cost savings may be realized.

Example 1 is a system for controlling delivery of electrostimulation to a patient spinal cord. The system comprises a data processor configured to receive a patient dataset representing a neural structure of at least a portion of the patient spinal cord, and extract a feature from the received patient dataset, and a programming control circuit configured to receive a computational spinal cord (CSC) model characterizing generic spinal cord anatomical and physical properties. The programming control circuit is configured to modify the CSC model to generate a patient-specific model based on the extracted feature, and compute a stimulation parameter value using the patient-specific model.

In Example 2, the subject matter of Example 1 optionally includes the CSC model that may include a finite-element method (FEM) model.

In Example 3, the subject matter of any one or more of Examples 1-2 optionally includes the CSC model that may characterize the spinal cord physical properties including one or more of spinal cord mechanical, electrical, optical, or chemical properties.

In Example 4, the subject matter of any one or more of Examples 1-3 optionally includes the CSC model that may include a lead model including information about position and orientation of electrodes on a stimulation lead relative to the patient spinal cord. The programming control circuit may be configured to compute, based at least in part on the lead model, a stimulation parameter value including polarities and current fractionalization of the electrodes on the stimulation lead.

In Example 5, the subject matter of Example 4 optionally includes the programming control circuit that may be configured to use the patient-specific model to identify locations of target current source poles, determine a target electric field using the locations of the target current source poles for activating one or more neural elements, and determine the polarities and the current fractionalization of the electrodes on the stimulation lead using the determined target electric field.

In Example 6, the subject matter of Example 5 optionally includes the programming control circuit that may be configured to determine the polarities and the current fractionalization by applying a transfer matrix to the target electric field.

In Example 7, the subject matter of Example 5 optionally includes the programming control circuit that may be configured to determine the polarities and the current fractionalization using a least-square fit of the target electric field.

In Example 8, the subject matter of Example 5 optionally includes the programming control circuit that may further be configured to determine a threshold stimulation amplitude based on the target electric field, where the threshold stimulation amplitude represents a minimal stimulation amplitude required to activate one or more neural elements.

In Example 9, the subject matter of any one or more of Examples 1-8 optionally includes the received patient dataset that may include a medical image of at least the portion of the patient spinal cord.

In Example 10, the subject matter of any one or more of Examples 1-9 optionally includes the received patient dataset that may characterize morphology of the neural structure of at least a portion of the patient spinal cord. The data processor may be configured to recognize a neural element of the patient spinal cord from the received patient dataset, and extract a geometric feature of the recognized neural element.

In Example 11, the subject matter of Example 10 optionally includes the recognized neural element that may include a representation of cerebrospinal fluid (CSF), and the geometric feature that may include a CSF thickness measurement.

In Example 12, the subject matter of Example 10 optionally includes the geometric feature that may include location or morphology of at least one of a dorsal root, a dorsal root ganglion, or a dorsal horn.

In Example 13, the subject matter of any one or more of Examples 1-12 optionally includes a posture sensor to detect a change in patient posture. The data processor may be configured to receive a first patient dataset before the detected posture change and a second dataset after the detected posture change, and to determine a change from a first feature extracted from the first patient dataset to a second feature extracted from the second patient dataset. The programming control circuit may be configured to modify the CSC model to generate the patient-specific model based on the determined change from the first extracted feature to the second extracted feature.

In Example 14, the subject matter of any one or more of Examples 1-13 optionally includes an ambulatory electrostimulator that may be configured to stimulate the patient spinal cord according to the computed stimulation parameter value.

In Example 15, the subject matter of Example 14 optionally includes an external device that may include the data processor and the programming control circuit. The external device may be configured to be communicatively coupled to the ambulatory electrostimulator and to program the ambulatory electrostimulator with the computed stimulation parameter value.

Example 16 is a method for operating a medical system to control delivery of electrostimulation. The method comprises steps of: receiving a patient dataset representing a neural structure of at least a portion of the patient spinal cord; extracting a feature from the received patient dataset via a data processor; and modifying a computational spinal cord (CSC) model via a programming control circuit to generate a patient-specific model based on the extracted feature, the CSC model characterizing spinal cord anatomical and physical properties; and computing, via the programming control circuit, a stimulation parameter value using the patient-specific model.

In Example 17, the subject matter of Example 16 optionally includes the received CSC model that may further include a lead model including information about position and orientation of electrodes on a stimulation lead relative to the patient spinal cord. The computed stimulation parameter value may include polarities and current fractionalization for the electrodes on the stimulation lead.

In Example 18, the subject matter of Example 17 optionally includes identifying locations of target current source poles using the extracted feature, determining a target electric field using the locations of the target current source poles for activating one or more neural elements, and determining the polarities and the current fractionalization of the electrodes on the stimulation lead using the determined target electric field.

In Example 19, the subject matter of any one or more of Examples 16-18 optionally includes the received patient dataset that may include a medical image of at least the portion of the patient spinal cord, and the extracted feature that may include a geometric feature of a neural element extracted from the medical image.

In Example 20, the subject matter of any one or more of Examples 16-19 optionally includes the geometric feature that may include at least one of: a cerebrospinal fluid thickness measurement; location or morphology of a dorsal root; location or morphology of a dorsal horn; or location or morphology of a dorsal root ganglion.

In Example 21, the subject matter of any one or more of Examples 16-20 optionally includes, programming an ambulatory electrostimulator with the computed stimulation parameter value via an external device, and delivering spinal cord stimulation according to the programmed stimulation parameter value via the ambulatory electrostimulator.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various examples discussed in the present document. The drawings are for illustrative purposes only and may not be to scale.

FIG. 1 illustrates, by way of example and not limitation, a block diagram of a neurostimulation system.

FIG. 2 illustrates, by way of example and not limitation, a block diagram of a stimulation device and a lead system that may implemented in the neurostimulation system of FIG. 1.

FIG. 3 illustrates, by way of example and not limitation, a block diagram of a programming device that may be implemented in the neurostimulation system of FIG. 1.

FIG. 4 illustrates, by way of example and not limitation, a diagram of an implantable pulse generator (IPG) and an implantable lead system, such as an example implementation of the stimulation device and lead system of FIG. 2.

FIG. 5 illustrates, by way of example and not limitation, a diagram of an implantable neurostimulation system and portions of an environment in which the system may be used.

FIG. 6 illustrates, by way of example and not limitation, a diagram of portions of a neurostimulation system.

FIG. 7 illustrates, by way of example and not limitation, a block diagram of an implantable stimulator and one or more leads of an implantable neurostimulation system, such as the implantable neurostimulation system of FIG. 6.

FIG. 8 illustrates, by way of example and not limitation, a block diagram of an external programming device of an implantable neurostimulation system, such as the implantable neurostimulation system of FIG. 6.

FIG. 9 illustrates, by way of example and not limitation, a block diagram of a portion of the system to modify a computational spinal cord (CSC) model to generate patient-specific stimulation parameters.

FIGS. 10A-10B illustrate, by way of example and not limitation, views of a numerical CSC model created using finite-element method (FEM).

FIGS. 11A-11C illustrates, by way of example and not limitation, patient images and morphological features extracted therefrom for used to generate a patient-specific CSC model.

FIG. 12 illustrates, by way of example and not limitation, a flow chart of a method for controlling delivery of electrostimulation to specific tissue of a patient.

FIG. 13 illustrates, by way of example of not limitation, a block diagram of an example machine upon which any one or more of the techniques discussed herein may perform.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses, among other things, systems and methods for controlling delivery of electrostimulation to a patient spinal cord using a patient-specific computational spinal cord model. An embodiment of a system comprises a data processor that can receive a patient dataset representing a neural structure of at least a portion of the patient spinal cord, and extract a feature from the received patient dataset. The patient dataset may include a medical image of the patient spinal cord, and the extracted feature may include one or more geometric features or measurements of a spinal structure. The system includes a programming control circuit to receive a computational spinal cord (CSC) model that characterizes generic spinal cord anatomical and physical properties. Using the extracted feature, the programming control circuit may modify the CSC model to generate a patient-specific model, and compute a stimulation parameter value using the patient-specific model. The system includes a programmer device to program the stimulation parameter value into an ambulatory electrostimulator, which can generate spinal cord stimulation according to the stimulation parameter value.

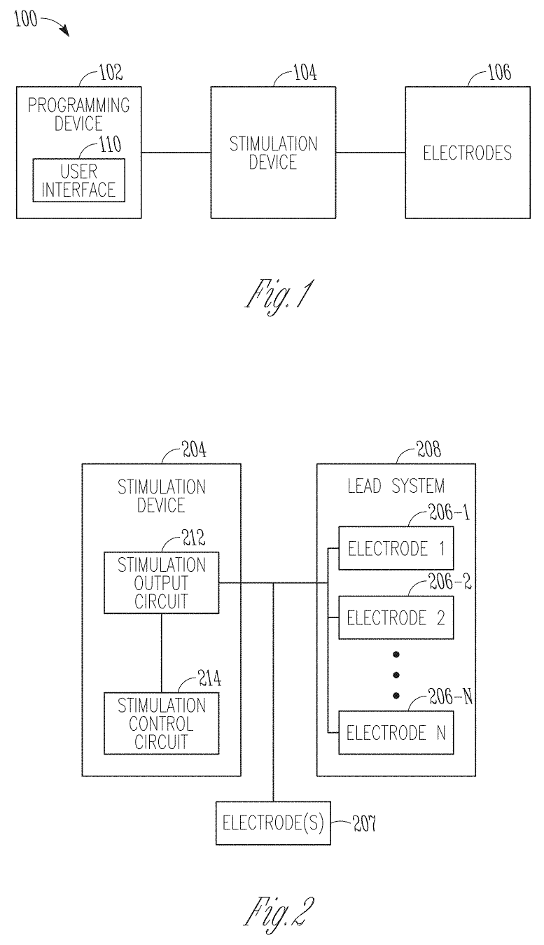

FIG. 1 illustrates, by way of example and not limitation, a block diagram of a neurostimulation system 100 configured for neurostimulation applications, including but not limited to SCS, DBS, PNS, and FES applications. The neurostimulation system 100 includes electrodes 106, a stimulation device 104, and a programming device 102. The electrodes 106 are configured to be placed on or near one or more neural targets in a patient. The stimulation device 104 is configured to be electrically connected to the electrodes 106 and can deliver neurostimulation energy, such as in the form of electrical pulses, to the one or more neural targets through the electrodes 106. Delivery of the neurostimulation may be controlled by using a plurality of stimulation parameters, such as parameters specifying a pattern of electrical pulses (pulse amplitude, pulse width, pulse frequency, pulse waveform, duty cycle, or pacing duration, among other parameters) and a selection of electrodes through which each of the electrical pulses is delivered. In various examples, at least some parameters of the plurality of stimulation parameters may be programmable by a user operating the system 100. In various examples, the stimulation device 104 may be configured to provide neurostimulation using an energy modality in addition to or in lieu of electrical stimulation, such as one of thermal, optical (e.g., laser), electromagnetic, chemical, or mechanical neural stimulation, or a hybrid stimulation using two or more different energy modalities.

The programming device 102 provides the user with accessibility to the user-programmable parameters. The programming device 102 may be configured to be communicatively coupled to stimulation device 104 via a wired or wireless link.

In this document, a “user” includes a physician or other clinician or caregiver who treats the patient using the system 100, and a “patient” includes a person who receives or is intended to receive neurostimulation delivered using the system 100. In some examples, the patient may be allowed to adjust his or her treatment using the system 100 to certain extent, such as by adjusting certain therapy parameters and entering feedback and clinical effect information.

The programming device 102 may include a user interface 110 that allows the user to control the operation of the system 100 (e.g., delivery of neurostimulation), and to monitor performance of the system 100 and conditions of the patient including responses to the delivery of the neurostimulation. The user may control the operation of the system 100 by setting and/or adjusting values of the user-programmable parameters.

The user interface 110 may be graphical user interface (GUI) that allows the user to set and/or adjust the values of the user-programmable parameters by creating and/or editing graphical representations of various waveforms. Such waveforms may include, for example, a waveform representing a pattern of stimulation pulses to be delivered to the patient as well as individual waveforms that are used as building blocks of the pattern of stimulation pulses, such as the waveform of each pulse in the pattern of stimulation pulses. The GUI may also allow the user to set and/or adjust stimulation fields each defined by a set of electrodes through which one or more stimulation pulses represented by a waveform are delivered to the patient. The stimulation fields may each be further defined by the distribution of the current of each stimulation pulse in the waveform. In various examples, stimulation pulses for a stimulation period (such as the duration of a therapy session) may be delivered to multiple stimulation fields.

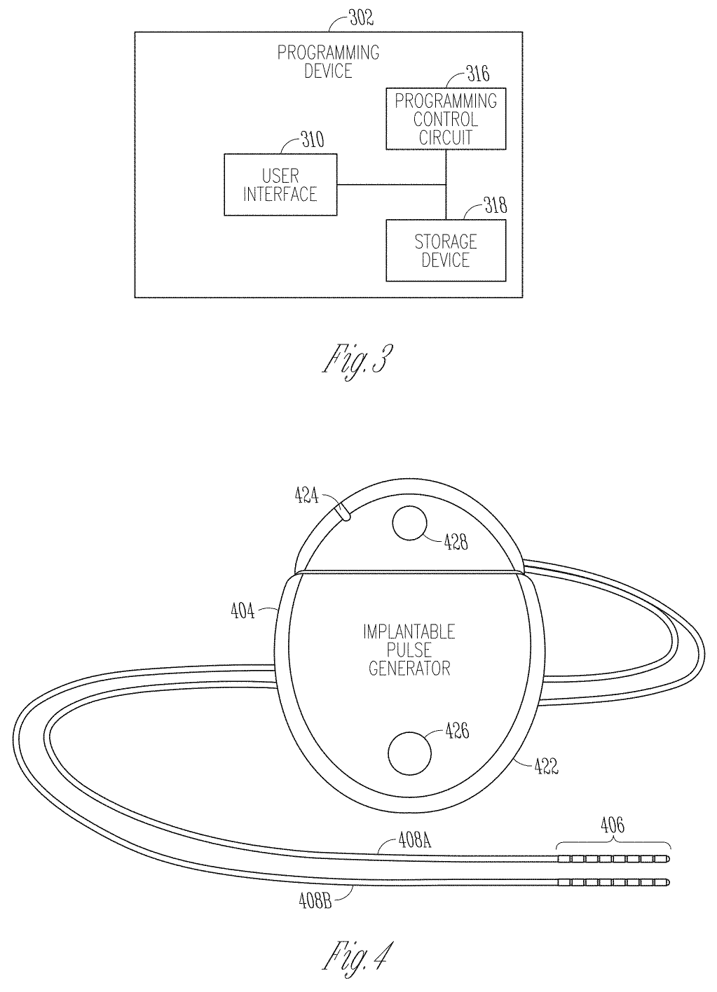

FIG. 2 illustrates, by way of example and not limitation, a block diagram of a stimulation device 204 and a lead system 208, such as may be implemented in the neurostimulation system 100. The stimulation device 204 may represent an example of the stimulation device 104 and includes a stimulation output circuit 212 and a stimulation control circuit 214. The stimulation output circuit 212 produces and delivers stimulation pulses. The stimulation control circuit 214 controls the delivery of the stimulation pulses from the stimulation output circuit 212 using the plurality of stimulation parameters, which specifies a pattern of stimulation pulses. The lead system 208 includes one or more leads each configured to be electrically connected to the stimulation device 204 and a plurality of electrodes 206 distributed in the one or more leads. The plurality of electrodes 206 includes electrode 206-1 through 206-N, each being a single electrically conductive contact providing for an electrical interface between stimulation output circuit 212 and tissue of the patient. The stimulation pulses are each delivered from stimulation output circuit 212 through a set of electrodes selected from the electrodes 206. In some examples, the stimulation pulses may include one or more individually defined pulses, and the set of electrodes may be individually definable by the user for each of the individually defined pulses or each of collections of pulse intended to be delivered using the same combination of electrodes. In some examples, one or more additional electrodes 207 (each of which may be referred to as a reference electrode) may be electrically connected to the stimulation device 204, such as one or more electrodes each being a portion of or otherwise incorporated onto a housing of the stimulation device 204. Monopolar stimulation uses a monopolar electrode configuration with one or more electrodes selected from the electrodes 206 and at least one electrode from the electrode(s) 207. Bipolar stimulation uses a bipolar electrode configuration with two electrodes selected from the electrodes 206 and none of the electrode(s) 207. Multipolar stimulation uses a multipolar electrode configuration with multiple electrodes selected from the electrodes 206, or multiple electrodes selected from the electrode(s) 207. The number of leads and the number of electrodes on each lead may depend on, for example, the distribution of target(s) of the neurostimulation and the need for controlling the distribution of electric field at each target. In one example, the lead system 208 includes two leads each having eight electrodes. In another example, the lead system 208 includes two leads each having sixteen electrodes.

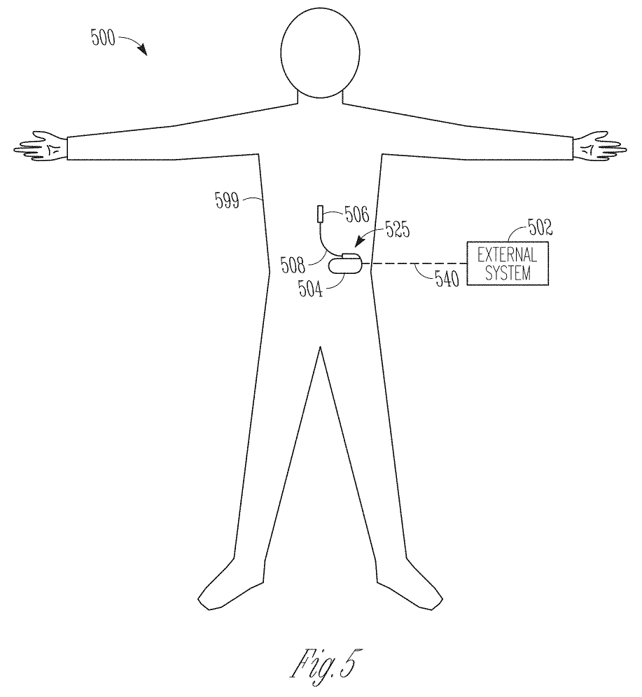

FIG. 3 illustrates, by way of example and not limitation, a block diagram of a programming device 302, such as may be implemented in neurostimulation system 100. The programming device 302, which is an example of the programming device 102, includes a storage device 318, a programming control circuit 316, and a user interface 310. The programming control circuit 316 may generate a plurality of stimulation parameters that controls the delivery of the stimulation pulses according to a specified stimulation program that may define, for example, stimulation waveform and electrode configuration. The user interface 310 may represent an example of the user interface 110. The storage device 318 stores, among other things, information used by programming control circuit 316, such as information about a stimulation device that relates the stimulation program to the plurality of stimulation parameters and information relating the stimulation program to a volume of activation in the patient. In some examples, the programming control circuit 316 may be configured to support one or more functions allowing for programming of stimulation devices, such as the stimulation device 104 including but not limited to its various examples as discussed in this document.

The storage device 318 may store a computational spinal cord (CSC) model that characterizes spinal cord anatomical and physical properties. The anatomical properties may include size, shape, or other geometric metrics of the spinal cord and various neural structures therein. The physical properties may include spinal cord mechanical, electrical, optical, or chemical properties. The CSC model may be a generic spinal cord model created using spinal cord data generalized from a patient population. The CSC model may be in a form of a parametric model, a statistical model, a shape-based model, or a volumetric model, among others. The CSC model may be a numerical model or an analytical model. In an example, the CSC model is a finite element method (FEM) spinal cord model. In another example, the CSC model is an approximation of a FEM spinal cord model, such that the output in regions of interest of an FEM model (e.g., voltages near electrodes) are represented by an analytical model.

The user interface 310 may receive from a user a patient dataset representing one or more neural structures of at least a portion of the patient spinal cord. In some examples, the patient dataset may be received from a patient database, such as an electronic medical record system or other storage devices. In an example, the patient dataset may characterize size, shape, morphology, or other geometric characteristics of the neural structures of the patient spinal cord. The programming control circuit 316 may modify the CSC model stored in the storage device 318 using the features extracted from the patient dataset, generate a patient-specific model, and compute a stimulation parameter value using the patient-specific model.

In some examples, the user interface 310 may allow for definition of a pattern of stimulation pulses for delivery during a neurostimulation therapy session by creating and/or adjusting one or more stimulation waveforms using a graphical method. The definition may also include definition of one or more stimulation fields each associated with one or more pulses in the pattern of stimulation pulses. As used in this document, a “stimulation program” may include the pattern of stimulation pulses including the one or more stimulation fields, or at least various aspects or parameters of the pattern of stimulation pulses including the one or more stimulation fields. In some examples, user interface 310 includes a GUI that allows the user to define the pattern of stimulation pulses and perform other functions using graphical methods. In this document, “neurostimulation programming” may include the definition of the one or more stimulation waveforms, including the definition of one or more stimulation fields.

In various examples, circuits of the neurostimulation 100, including but not limited to its various examples discussed in this document, may be implemented using a combination of hardware and software. For example, the circuit of user interface 110, stimulation control circuit 214, and programming control circuit 316, including but not limited to their various examples discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

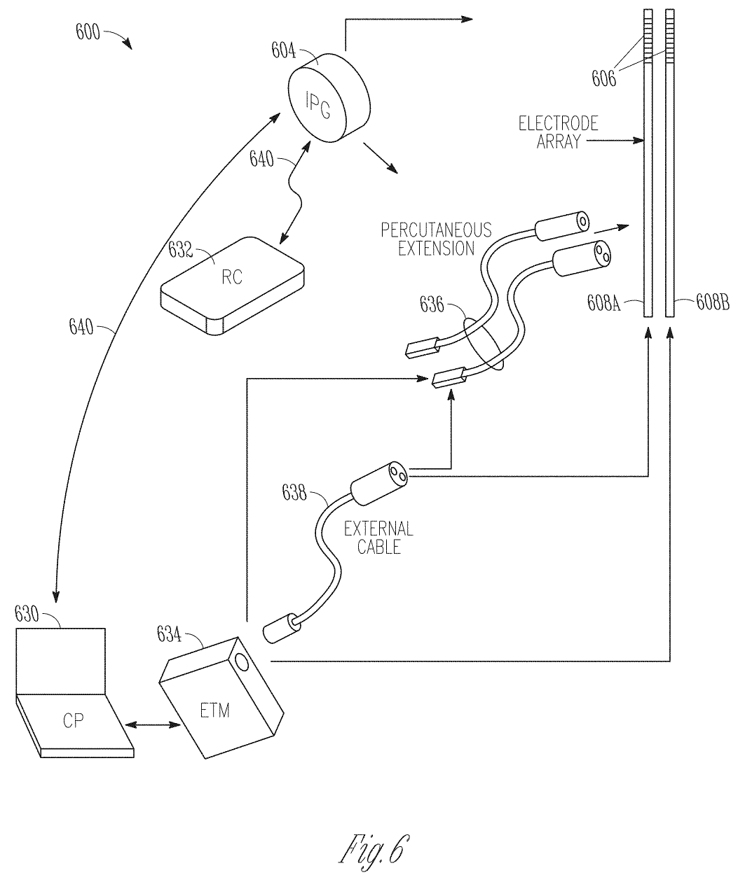

FIG. 4 illustrates, by way of example and not limitation, a diagram of an implantable pulse generator (IPG) 404 and implantable leads 408A-408B. The IPG 404 represents an example of stimulation device 204. The implantable leads 408A-408B represents an example of the lead system 208. As illustrated in FIG. 4, the IPG 404 may be coupled to the implantable leads 408A-408B at a proximal end of each lead. The distal end of each lead includes electrical contacts or electrodes 406 for contacting a tissue site targeted for electrical neurostimulation. As illustrated in FIG. 4, the leads 408A-408B each include eight electrodes 406 at the distal end. The number and arrangement of leads 408A-408B and electrodes 406 as shown in FIG. 4 are only an example, and other numbers and arrangements are possible. In various examples, the electrodes are ring electrodes. The implantable leads and electrodes may be configured by shape and size to provide electrical neurostimulation energy to a neuronal target included in the subject's brain, or configured to provide electrical neurostimulation energy to a nerve cell target included in the subject's spinal cord.

The IPG 404 may include a hermetically sealed IPG case 422 to house the electronic circuitry of IPG 404, an electrode 426 formed on the IPG case 422, and an IPG header 424 for coupling the proximal ends of the leads 408A-408B. The IPG header 424 may optionally include an electrode 428. The electrodes 426 and/or 428 represent examples of electrode(s) 207 and may each be referred to as a reference electrode. Neurostimulation energy may be delivered in a monopolar (also referred to as unipolar) mode using the electrodes 426 or 428 and one or more electrodes selected from the electrodes 406. Neurostimulation energy may be delivered in a bipolar mode using a pair of electrodes of the same lead (lead 408A or 408B). Neurostimulation energy may be delivered in an extended bipolar mode using one or more electrodes of a lead (e.g., one or more electrodes of lead 408A) and one or more electrodes of a different lead (e.g., one or more electrodes of lead 408B).

The electronic circuitry of IPG 404 may include a control circuit that controls delivery of the neurostimulation energy. The control circuit may include a microprocessor, a digital signal processor, application specific integrated circuit (ASIC), or other type of processor, interpreting or executing instructions included in software or firmware. The neurostimulation energy may be delivered according to specified (e.g., programmed) modulation parameters. Examples of setting modulation parameters may include, among other things, selecting the electrodes or electrode combinations used in the stimulation, configuring an electrode or electrodes as the anode or the cathode for the stimulation, specifying the percentage of the neurostimulation provided by an electrode or electrode combination, and specifying stimulation pulse parameters. Examples of pulse parameters include, among other things, the amplitude of a pulse (specified in current or voltage), pulse duration (e.g., in microseconds), pulse rate (e.g., in pulses per second), and parameters associated with a pulse train or pattern such as burst rate (e.g., an “on” modulation time followed by an “off” modulation time), amplitudes of pulses in the pulse train, polarity of the pulses, etc.

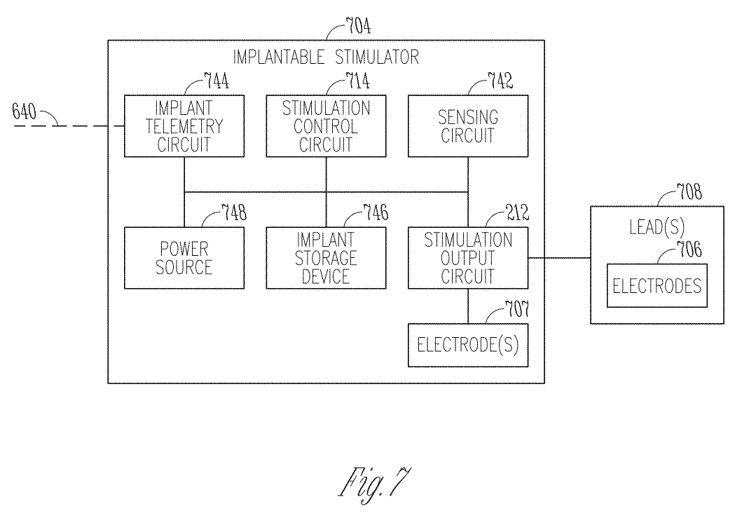

FIG. 5 illustrates, by way of example and not limitation, a diagram of an implantable neurostimulation system 500 and portions of an environment in which system 500 may be used. The system 500 includes an implantable system 525, an external system 502, and a telemetry link 540 providing for wireless communication between implantable system 525 and external system 502. Implantable system 525 is illustrated in FIG. 5 as being implanted in the patient body 599.

The implantable system 525 includes an implantable stimulator (also referred to as an IPG) 504 representing an example of the IPG 404 or the stimulation device 204, a lead system 508 representing an example of one or more of leads 408A-408B or the lead system 208, and electrodes 506 representing an example of electrodes 206. In the illustrated example, implantable lead system 508 is arranged to provide SCS to a patient, with the stimulation target being neuronal tissue in the patient spinal cord. In various examples, the present subject matter may be applied to neurostimulation of any types and targets, including but not limited to SCS, DBS, PNS, and FES.

The external system 502 may represent an example of the programming device 302. In various examples, the external system 502 may include one or more external (non-implantable) devices each allowing the user and/or the patient to communicate with the implantable system 525. In some examples, the external system 502 includes a programming device intended for the user to initialize and adjust settings for the implantable stimulator 504 and a remote-control device intended for use by the patient. For example, the remote-control device may allow the patient to turn on or off the implantable stimulator 404, and/or to adjust certain patient-programmable parameters of the plurality of stimulation parameters.

The sizes and shapes of the elements of the implantable system 525 and their location in patient body 599 are illustrated by way of example and not by way of restriction. An implantable system is discussed as a specific application of the programming according to various examples of the present subject matter. In various examples, the present subject matter may be applied in programming any type of stimulation device that uses electrical pulses as stimuli, regarding less of stimulation targets in the patient body and whether the stimulation device is implantable.

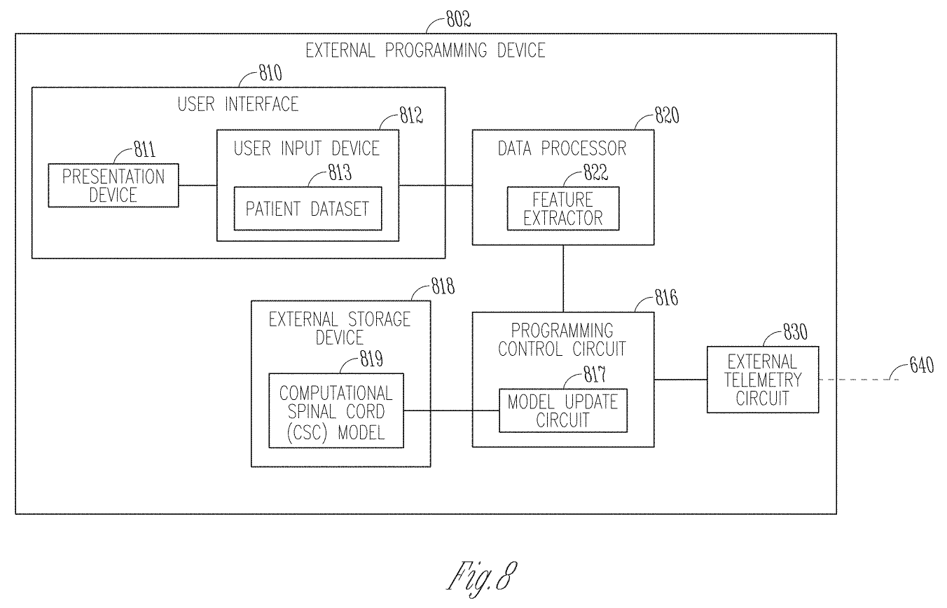

FIG. 6 illustrates, by way of example and not limitation, a diagram of portions of a neurostimulation system 600. The system 600 includes an IPG 604, implantable neurostimulation leads 608A-608B, an external remote controller (RC) 632, a clinician's programmer (CP) 630, and an external trial modulator (ETM) 634. The IPG 404 may be electrically coupled to the leads 608A-608B directly or through the percutaneous extension leads 636. The ETM 634 may be electrically connectable to leads 608A-608B via one or both of the percutaneous extension leads 636 and/or the external cable 638. The system 600 may represent an example of the system 100, with IPG 604 representing an example of the stimulation device 104, electrodes 606 of leads 608A-608B representing electrodes 106, and CP 630, RC 632, and ETM 634 collectively representing programming device 102.

The ETM 634 may be standalone or incorporated into the CP 630. The ETM 634 may have similar pulse generation circuitry as the IPG 604 to deliver neurostimulation energy according to specified modulation parameters as discussed above. The ETM 634 is an external device that is typically used as a preliminary stimulator after leads 408A-408B have been implanted and used prior to stimulation with the IPG 604 to test the patient responsiveness to the stimulation that is to be provided by the IPG 604. Because the ETM 634 is external it may be more easily configurable than the IPG 604.

The CP 630 may configure the neurostimulation provided by the ETM 634. If the ETM 634 is not integrated into the CP 630, the CP 630 may communicate with the ETM 634 using a wired connection (e.g., over a USB link) or by wireless telemetry using a wireless communications link 640. The CP 630 may also communicate with the IPG 604 using the wireless communications link 640.

An example of wireless telemetry is based on inductive coupling between two closely placed coils using the mutual inductance between these coils. This type of telemetry is referred to as inductive telemetry or near-field telemetry because the coils must typically be closely situated for obtaining inductively coupled communication. The IPG 604 may include the first coil and a communication circuit. The CP 630 may include or otherwise electrically connected to the second coil such as in the form of a wand to be placed near the IPG 604. Another example of wireless telemetry includes a far-field telemetry link, also referred to as a radio frequency (RF) telemetry link. A far-field, also referred to as the Fraunhofer zone, refers to the zone in which a component of an electromagnetic field produced by the transmitting electromagnetic radiation source decays substantially proportionally to 1/r, where r is the distance between an observation point and the radiation source. Accordingly, far-field refers to the zone outside the boundary of r=λ/2 π, where λ is the wavelength of the transmitted electromagnetic energy. In one example, a communication range of an RF telemetry link is at least six feet but may be as long as allowed by the particular communication technology. RF antennas may be included, for example, in the header of the IPG 604 and in the housing of the CP 630, eliminating the need for a wand or other means of inductive coupling. An example is such an RF telemetry link is a Bluetooth® wireless link.

The CP 630 may be used to set modulation parameters for the neurostimulation after the IPG 604 has been implanted. This allows the neurostimulation to be tuned if the requirements for the neurostimulation change after implantation. The CP 630 may also upload information from the IPG 604.

The RC 632 also communicates with the IPG 604 using a wireless link 340. The RC 632 may be a communication device used by the user or given to the patient. The RC 632 may have reduced programming capability compared to the CP 630. This allows the user or patient to alter the neurostimulation therapy but does not allow the patient full control over the therapy. For example, the patient may be able to increase the amplitude of stimulation pulses or change the time that a preprogrammed stimulation pulse train is applied. The RC 632 may be programmed by the CP 630. The CP 630 may communicate with the RC 632 using a wired or wireless communications link. In some examples, the CP 630 is capable of programming the RC 632 when remotely located from the RC 632.

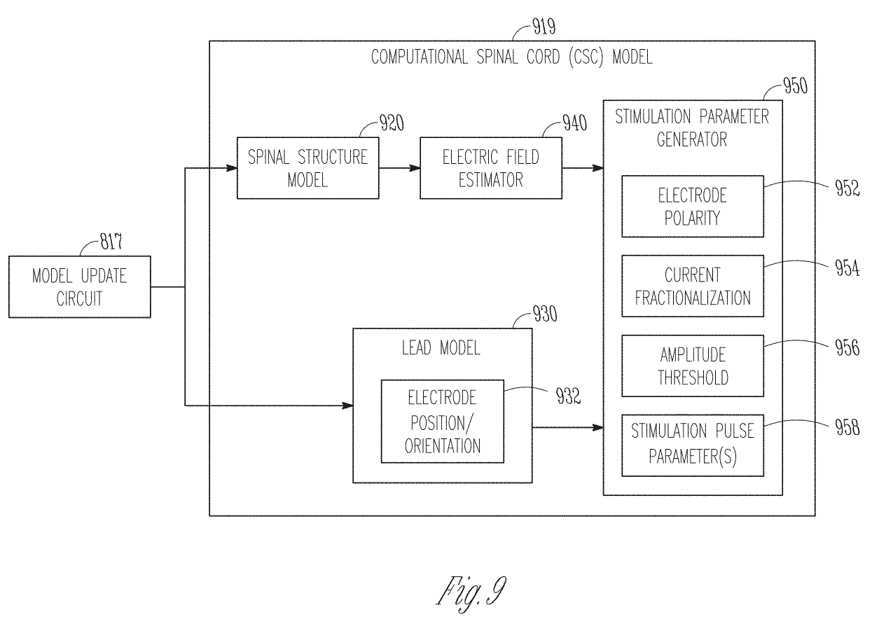

FIG. 7 illustrates, by way of example and not limitation, a block diagram of an implantable stimulator 704 and one or more leads 708 of an implantable neurostimulation system, such as the implantable system 600. The implantable stimulator 704 may represent an example of the stimulation device 104 or 204 and may be implemented, for example, as IPG 404. The lead(s) 708 may represent an example of the lead system 208 and may be implemented, for example, as implantable leads 408A-408B. The lead(s) 708 includes electrodes 706, which may represent an example of the electrodes 106 or 206 and may be implemented as the electrodes 406.

The implantable stimulator 704 may include a sensing circuit 742, a stimulation output circuit 212, a stimulation control circuit 714, an implant storage device 746, an implant telemetry circuit 744, a power source 748, and one or more electrodes 707. The sensing circuit 742 may in some examples be optional and required only when the stimulator needs a sensing capability. When included, the sensing circuit 742 senses one or more physiological signals for purposes of patient monitoring and/or feedback control of the neurostimulation. Examples of the one or more physiological signals include neural and other signals each indicative of a condition of the patient that is treated by the neurostimulation and/or a response of the patient to the delivery of the neurostimulation. The stimulation output circuit 212 is electrically connected to the electrodes 706 through one or more leads 708 as well as the electrodes 707, and delivers each of the stimulation pulses through a set of electrodes selected from the electrodes 706 and the electrode(s) 707. The stimulation control circuit 714 may represent an example of the stimulation control circuit 214 and controls the delivery of the stimulation pulses using the plurality of stimulation parameters specifying the pattern of stimulation pulses. In one example, the stimulation control circuit 714 controls the delivery of the stimulation pulses using the one or more sensed physiological signals. The implant telemetry circuit 744 provides the implantable stimulator 704 with wireless communication with another device, such as the CP 630 and the RC 632, including receiving values of the plurality of stimulation parameters from the other device. The implant storage device 746 stores values of the plurality of stimulation parameters, such as stimulation parameters generated by an external programming device (e.g., the CP 630 or the RC 632) in the external system 502. The power source 748 provides the implantable stimulator 704 with energy for its operation. In one example, the power source 748 includes a battery. In one example, the power source 748 includes a rechargeable battery and a battery charging circuit for charging the rechargeable battery. The implant telemetry circuit 744 may also function as a power receiver that receives power transmitted from an external device through an inductive couple. The electrode(s) 707 allow for delivery of the stimulation pulses in the monopolar mode. Examples of the electrode(s) 707 include the electrode 426 and the electrode 418 in the IPG 404 as illustrated in FIG. 4.

In one example, the implantable stimulator 704 is used as a master database. A patient implanted with the implantable stimulator 704 (such as may be implemented as the IPG 604) may therefore carry patient information needed for his or her medical care when such information is otherwise unavailable. The implant storage device 746 is configured to store such patient information. For example, the patient may be given a new RC 632 and/or travel to a new clinic where a new CP 630 is used to communicate with the device implanted in him or her. The new RC 632 and/or CP 630 may communicate with the implantable stimulator 704 to retrieve the patient information stored in the implant storage device 746 through the implant telemetry circuit 744 and the wireless communication link 640, and allow for any necessary adjustment of the operation of the implantable stimulator 704 based on the retrieved patient information. In various examples, the patient information to be stored in the implant storage device 746 may include, for example, positions of lead(s) 708 and electrodes 706 relative to the patient anatomy (e.g., anatomy of patient spinal cord), clinical effect map data, objective measurements using quantitative assessments of symptoms (for example using micro-electrode recording, accelerometers, and/or other sensors), and/or any other information considered important or useful for providing adequate care for the patient. In various examples, the patient information to be stored in the implant storage device 746 may include data transmitted to the implantable stimulator 704 for storage as part of the patient information and data acquired by the implantable stimulator 704, such as by using the sensing circuit 742.

In various examples, the sensing circuit 742 (if included), the stimulation output circuit 212, the stimulation control circuit 714, the implant telemetry circuit 744, the implant storage device 746, and the power source 748 are encapsulated in a hermetically sealed implantable housing or case, and the electrode(s) 707 are formed or otherwise incorporated onto the case. In various examples, the lead(s) 708 are implanted such that the electrodes 706 are placed on and/or around one or more targets to which the stimulation pulses are to be delivered, while the implantable stimulator 704 is subcutaneously implanted and connected to the lead(s) 708 at the time of implantation.

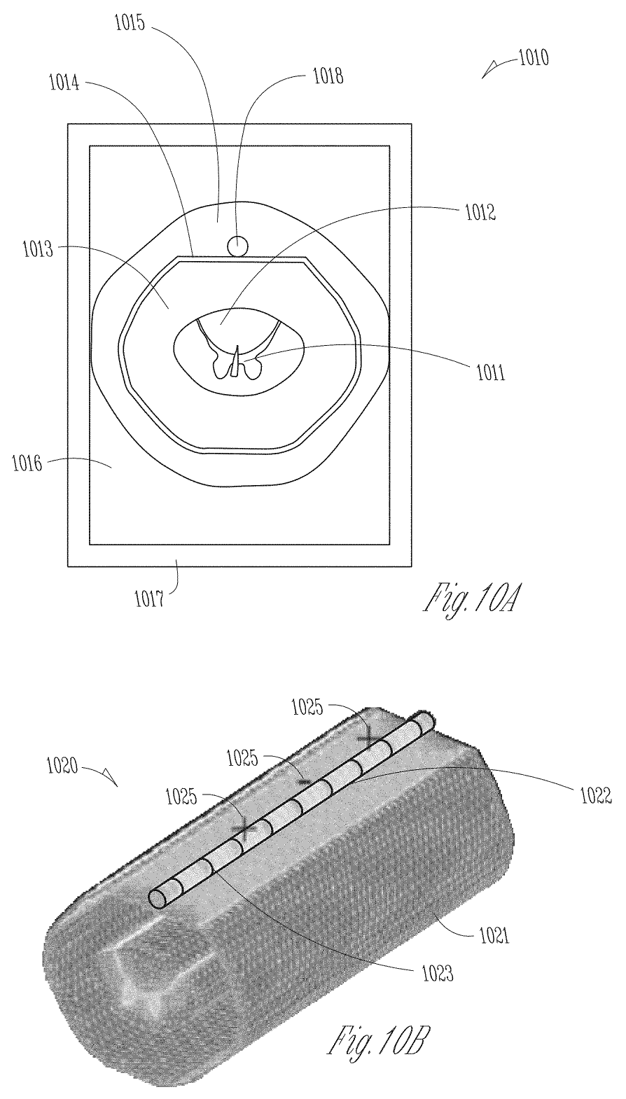

FIG. 8 illustrates, by way of example and not limitation, a block diagram of an external programming device 802 of an implantable neurostimulation system, such as the system 600. The external programming device 802 may represent an example of the programming device 102 or 302, and may be implemented, for example, as CP 630 and/or RC 632. In the example as illustrated in FIG. 8, the external programming device 802 includes a user interface 810, a data processor 820, a programming control circuit 816, an external storage device 818, and an external telemetry circuit 830.

The user interface 810, which represents an example of the user interface 310, may allow for use inputting patient medical data, among other monitoring and programming tasks. The user interface 810 includes a presentation device 811 and a user input device 812. The presentation device 811 may include any type of interactive or non-interactive display screens. The user input device 812 may include any type of user input devices that supports the various functions discussed in this document, such as touchscreen, keyboard, keypad, touchpad, trackball, joystick, and mouse. In one example, the user interface 810 includes a graphical user interface (GUI). The GUI may also allow the user to perform any functions discussed in this document where graphical presentation and/or editing are suitable as may be appreciated by those skilled in the art.

In an example, the user input device 812 may receive user input of a patient dataset 813 representing a neural structure of at least a portion of the spinal cord of a patient. In some examples, the patient dataset 813 may be received from a patient database, such as an electronic medical record system or other storage devices separated from and external to the external programming device 802. The patient dataset 813 may represent shape, appearance, size, morphology, or other geometric characteristics of one or more neural structures of the patient spinal cord. The patient dataset 813 may include an image, a point cloud, a parametric model, or other data formats. In an example, the received patient dataset 813 includes a medical image, such as an image obtained with fluoroscopy, magnetic resonance imaging (MRI), computed tomography (CT) scan, computed axial tomography (CAT) scan, Positron Emission Tomography (PET) scan, medical ultrasonography, or nuclear functional imaging, among other medical imaging modalities. The medial image may have a specific data or file format, such as Analyze, Neuroimaging Informatics Technology Initiative (Nifti), Minc, and Digital Imaging and Communications in Medicine (Dicom). In addition to the image data characterizing image pixels, the received patient dataset 813 may contain metadata. Metadata include information associated with the image beyond the pixel data, such as image matrix dimensions, the spatial resolution, the pixel depth, and the photometric interpretation. The metadata may be stored together with the image data in a single data file. Alternatively, the metadata and the image data may be stored in separate files. In various examples, graphical and/or textual presentations of the patient dataset 813 may be displayed on a screen of the presentation device 811. A user may manipulate the patient dataset 813 via the user input devices, such as by selecting, dragging, rotating, zooming, or otherwise editing the displayed medical image. In an example, a user may use the user input devices to perform measurements of various neural structures on the displayed medical image.

The data processor 820 may analyze the patient dataset 813 to recognize a target neural element of the patient spinal cord. By way of example and not limitation, the neural elements of the patient spinal cord may include gray matter, white matter, a dorsal root, a dorsal root ganglion (DRG), or cerebrospinal fluid (CSF), among others. Spinal structures other than neural elements may also be recognized, such as bony anatomy of the spinal cord including inter-disc space, vertebra, and intervertebral foramen, among other anatomical landmarks. The data processor 820 may recognize the target element using a template-matching method such as based on a similarity metric between a portion of the patient dataset 813 and a known morphological template of the target element. In some examples, the data processor 820 may recognize the target neural element, or classify the neural element of the patient spinal cord into one of a number of types of neural elements, using a machine-learning method, such as a neural network, clustering, a support vector machine, or a Bayesian network.

The data processor 820 may include a feature extractor 822 configured to extract a feature from the patient dataset 813, such as a feature associated with a recognized neural element. The extracted feature may include a morphological feature representing position, orientation, thickness, distance, angulation, or trajectory, among other geometric metrics of one or more neural structures of the patient spinal cord. The extracted feature may additionally include features take from other spinal structures (e.g., the bony anatomy of the spinal cord) including, for example, inter-disc space, vertebral size, and location and size of intervertebral foramen. In some examples, the feature extractor 822 may predict or generalize features of neural elements based on features of bony anatomy. For example, if patient dataset is a fluoroscopy image, then only bony anatomy features may be extracted (e.g., pedicle location or foramen location), and no neural elements may be effectively detected from the fluoroscopy image. The feature extractor 822 may predict neural element feature such as root trajectories based on pedicle location, or estimate DRG location based on foramen location.

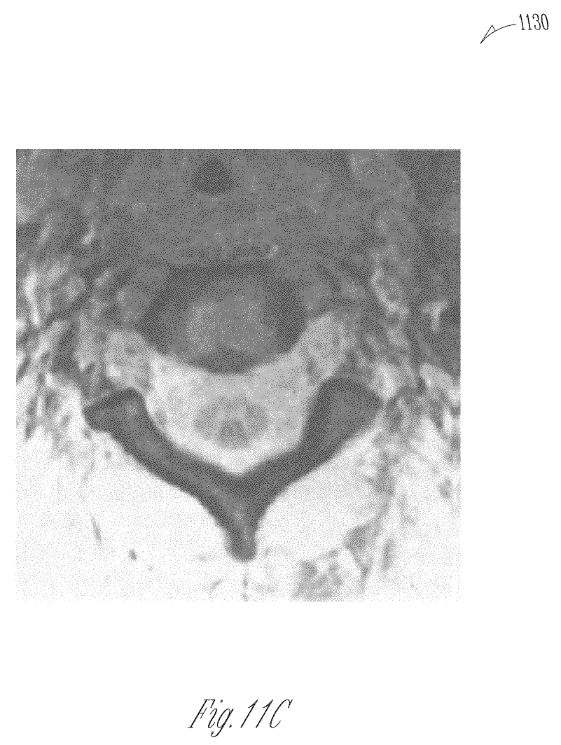

The extracted feature may be used to update a generic, population-based computational spinal cord (CSC) model 819, as to be discussed in the following. The type of the extracted feature may be dependent on the CSC model 819. For example, if the CSC model 819 determines stimulation parameters using input parameters such as the position, orientation, thickness, spatial distance, angulation, or other geometric metrics of neural elements, then the feature extractor 822 may accordingly extract features corresponding to the model input parameters. Examples of spinal cord dataset (e.g., a medical image) and extraction of morphological feature therefrom are discussed below, such as with reference to FIGS. 11A-11C.

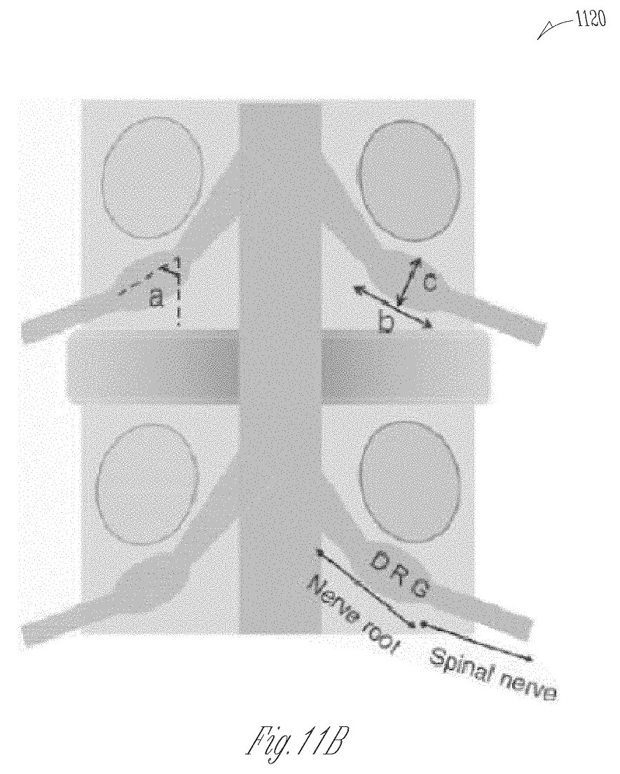

The external storage device 818, which may represent an example of the storage device 318, stores a computational spinal cord (CSC) model 819 that characterizes anatomical and physical properties of neural structures of patient spinal cord, as well as various parameters and building blocks for delivery of spinal cord stimulation. The anatomical properties may include size, shape, appearance, or other geometric metrics of the spinal cord and various neural structures therein. The physical properties may include spinal cord mechanical, electrical, optical, or chemical properties. The CSC model 819 may be a generic, population-based spinal cord model created based on spinal cord anatomy and physical properties generalized from a population. The generic CSC model 819 may be in a form of a parametric model, a statistical model, a shape-based model, or a volumetric model. In an example, the CSC model 819 is a numerical model, such as a finite element method (FEM) spinal cord model. In another example, the CSC model 819 is an analytical model. The analytical model may be an optimal fit to the FEM spinal cord model such as to satisfy a specified optimization criterion (e.g., fitting error falling below a specified threshold). The analytical model may be computationally efficient in determining stimulation parameters such as electric field strength. In various examples, in addition to modeling of the spinal structures, the CSC model 819 may further include a lead model including representation of a lead body, and position and orientation of electrodes on the lead relative to the patient spinal cord. Electrode positions and orientation may be used to determine stimulation parameters such as current fractionalization, total current, or electrode polarities. Examples of a numerical CSC model are discussed below, such as with reference to FIGS. 10A-10B.

The programming control circuit 816, which may represent an example of programming control circuit 316, generates one or more stimulation parameters that may be transmitted to and used by the implantable stimulator 704. The programming control circuit 816 is coupled to the data processor 820 and the external storage device 818, and includes a model update circuit 817 that is configured to modify the CSC model 819 to generate a patient-specific model based on the extracted feature from the patient dataset 813. Using the patient-specific model, the programming control circuit 816 may compute stimulation parameter values. Programming of stimulation parameter based on a patient-specific model, as discussed in this document, has several technological advantages over the conventional current steering methodology based on, for example, lead-specific navigation tables. By executing the patient-specific model, the programming control circuit 816 may implement a navigation paradigm that mimics arbitrary electric fields (e.g., a bipole with an arbitrary separation and/or arbitrary position relative to the electrode array), thereby de-linking the development of leads from substantial software changes (typically involving the development of new navigation tables) to expedite development of new lead technology. As a result, the model-based stimulation parameter optimization can be more computationally efficient and can potentially reduce technology development cost. Furthermore, as the patient-specific model takes into account inter-subject spinal structure differences and intra-subject variation in spinal structure over time or under different medical conditions, compared to a generic model, the patient-specific model may provide more individualized current steering and patient-specific stimulation parameter optimization.

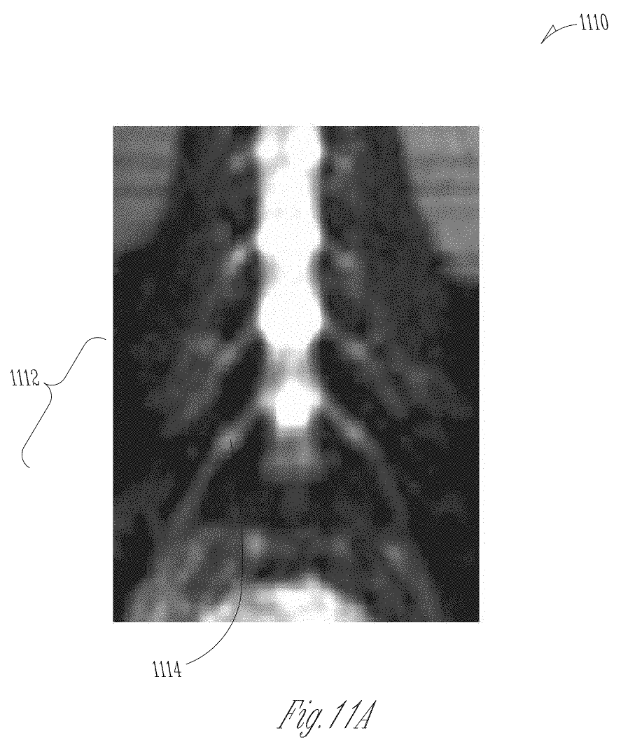

The CSC model 819 may accept relative electrode positions and a target electric field, and map the target electric field to the electrodes on a lead, thereby yielding polarities and the current fractionalization for the electrodes, among other stimulation parameters. For a specific patient, the model update circuit 817 may modify the CSC model 819 by adjusting, among other things, the target electric field and/or the relative electrode positions. The model adjustment may be based on the features or measurements of various patient-specific spinal structures taken from the patient dataset 813. For example, estimation of target electric field may be affected by position, orientation, angulation, trajectory, and other geometric metrics of one or more neural structures of the spinal cord. Through the mapping algorithm of the CSC model 819, the resultant stimulation parameters (e.g., total current, electrode polarities, and/or current fractionalization among the electrodes) are also patient-specific, and correspond to the anatomical and physical properties of the spinal cord of the specific patient. In various examples, the programming control circuit 816 may check values of the stimulation parameters against safety rules to limit these values within constraints of the safety rules. Examples of modifying the CSC model 819 and generating various patient-specific stimulation parameters are discussed below, such as with reference to FIG. 9.

In an example, the model update circuit 817 may modify the CSC model in response to a change in patient medical status, such as development of a new medical condition or progression of an existing medical condition. For example, spinal degeneration, such as degenerative disc disease, may affect the morphology and physical properties of some spinal structures. Patient data (e.g., medical images) may be taken after the identified medical condition. The feature extractor 822 may extract features from the dataset of the degenerated spinal cord, and the programming control circuit 816 may modify the CSC model to generate stimulation parameters corresponding to patient present medical condition.

In another example, modification of the CSC model may be triggered by a change in patient functional state, such as a change in patient posture. A posture change, such as from a supine position to a sitting or standing position, may alter relative location, morphology, trajectory, or other geometric metrics of one or more spinal structures (e.g., dorsal column, a dorsal horn, a dorsal root, or a dorsal root ganglion), thereby affecting the electric field distribution. Parameters for spinal cord stimulation (e.g., electrode polarities, total current, current fractionalization, and/or stimulation intensity parameters) prior to the posture change may not provide desired therapeutic outcome after the posture change. To compensate for the posture-related positional and morphological changes of spinal structures, the model update circuit 817 may update the CSC model for different patient postures. In an example, the patient dataset 813 may include several datasets each corresponding to a specific posture, such as a medical image respectively taken at supine, sitting, or standing postures. In an example, the datasets corresponding to various postures may be pre- generated and stored in a patient database, such as an electronic medical record system. The feature extractor 822 may extract, from each posture-indicated dataset, respective posture-indicated features including, for example, location, trajectory, or morphology of various spinal structures. When there is an indication of patient posture change (e.g., manually provided by the patient, or automatically detected by a posture sensor), the data processor circuit 820 may determine a change of posture-indicated features before and after the posture change. The model update circuit 817 may modify the CSC model 819 to generate the patient-specific model based on the determined feature change corresponding to the posture change. In an example, a posture sensor may be included in the implantable stimulator 704 to detect a change in patient posture. Examples of the posture sensor include a tilt switch, an accelerometer, or an impedance sensor. The patient posture change-based model update as described herein may be triggered by the detected posture change.

The external telemetry circuit 830 provides external programming device 802 with wireless communication with another device such as implantable stimulator 704 via wireless communication link 640, including transmitting the plurality of stimulation parameters to implantable stimulator 704 and receiving information including the patient data from implantable stimulator 704. In one example, external telemetry circuit 830 also transmits power to implantable stimulator 704 through an inductive couple. In various examples, wireless communication link 640 may include an inductive telemetry link (near-field telemetry link) and/or a far-field telemetry link (RF telemetry link). External telemetry circuit 830 and implant telemetry circuit 744 each include an antenna and RF circuitry configured to support such wireless telemetry.

In various examples, the external programming device 802 may have operation modes including a composition mode and a real-time programming mode. Under the composition mode (also known as the pulse pattern composition mode), user interface 810 is activated, while programming control circuit 816 is inactivated. Programming control circuit 816 does not dynamically update values of the plurality of stimulation parameters in response to any change in the one or more stimulation waveforms. Under the real-time programming mode, both user interface 810 and programming control circuit 816 are activated. Programming control circuit 816 dynamically updates values of the plurality of stimulation parameters in response to changes in the set of one or more stimulation waveforms, and transmits the plurality of stimulation parameters with the updated values to implantable stimulator 704.

FIG. 9 illustrates, by way of example and not limitation, a block diagram of a portion of the system to modify a CSC model 919 to generate patient-specific stimulation parameters. The CSC model 919, which represents an example of the CSC model 819, may include a spinal structure model 920 and a lead model 930. The spinal structure model 920 is a generic, population-based spinal cord model created based on information of spinal anatomy and physical properties generalized from a patient population. The lead model 930 includes representation of a stimulation lead, and position and/or orientation 932 of the lead electrodes relative to the patient spinal cord. Exemplary spinal structure model 920 and the lead model 930 based on FEM methods are illustrated in FIGS. 10A-10B.

The CSC model 919 comprises an electric field estimator 940 configured to estimate an electric field for activating one or more neural elements. The field estimation may be based on a target current source pole configuration. The target current source poles are imaginary configuration of electrode locations with respective polarities. The target current source poles, arranged in the identified configuration, may generate an electric field for stimulating various neural elements of the patient spinal cord. Electric potential, created by the target current source poles on imaginary contacts, is produced by a linear combination of fractionalized current on physically available electrodes. The target current source pole configuration includes polarity (i.e., anode or cathode), location, and current strength of target current source poles. In various examples, the target current source poles may be configured as monopole, bipole, tripole, or other multipole configurations. By way of example and not limitation, a multipole configuration may include a center cathode and two or more anodes surrounding the center cathode.

Estimation of target electric field from the identified target current source pole configuration may be affected by anatomical and physical properties of various spinal structures, as provided by the spinal structure model 920. As illustrated in FIG. 9, the model update circuit 817 may modify the spinal structure model 920 using the features extracted from the patient dataset 813. In an example, the extracted feature includes a thickness measurement of a dorsal cerebrospinal fluid (CSF). In another example, the extracted feature includes location of a dorsal root of at least the portion of patient spinal cord. The location of a dorsal root may be represented relative to the electrodes of the stimulation lead. The extracted feature may further include morphology of a dorsal root such as relative to the electrodes of the stimulation lead. In yet another example, the geometric feature includes location or morphology of a dorsal root ganglion, such as an angulation, a length, or a width of dorsal root ganglia. In another example, the extracted feature includes location or morphology of a dorsal horn, such as relative to the electrodes of the stimulation lead.

The stimulation parameter generator 950 may generate one or more stimulation parameters using the locations of target current source poles from the electric field estimator 940 and the electrode positions information 932. By way of example and not limitation, the stimulation parameters include electrode polarity 952 for the electrodes on the stimulation lead, current fractionalization 954, a threshold stimulation amplitude 956, and one or more stimulation pulse parameters 956.

The stimulation parameter generator 950 may determine the locations of target current source poles relative to the electrodes, and model an electric field generated by the target current source poles to determine desired field potential values at a plurality of spatial observation points. The locations of the target current source poles may be determined in a manner that places the resulting electric field over an identified region of the patient to be stimulated. In an example, the spatial observation points are spaced in a manner that would cover the entire tissue region to be stimulated and/or a tissue region that should not be stimulated. The locations of the target current source poles may be defined by the user, and may be displayed to the user along with the electrode locations, which as briefly discussed above, may be determined based on electrical measurements taken at the electrodes. In an example, the target current source poles may be selected in a manual screen (e.g., by using a mouse to click around the electrode array), or the target current sources poles may be selected using a navigation screen (e.g., by steering the target current source poles around the electrodes using directional controls, such as by manipulating displayed arrow keys or by using a joystick). Further details describing the use of manual screens and navigation screens are set forth in U.S. Patent Application No. 61/080,187, entitled “SYSTEM AND METHOD FOR CONVERTING TISSUE STIMULATION PROGRAMS IN A FORMAT USABLE BY AN ELECTRICAL CURRENT NAVIGATOR,” which is incorporated herein by reference in its entirety. Alternatively, the stimulation parameter generator 950 may automatically determine the locations of the target current source poles, e.g., heuristically (e.g., cathodes or anodes located where stimulation is to occur or not to occur) or using a model-based evaluation (e.g., an activating function fit). The number of spatial observation points should be selected to provide a reasonably accurate modeling of the ideal electric field without requiring an inordinate amount of processing time and/or computer resources. For example, the number of spatial observation points may be proportional to the number of electrodes and may number on the order of several thousand. Locations of target current source poles may be selected based on types of the neural structures. For example, the target current source poles that are optimal to stimulate dorsal roots, dorsal horn elements, and dorsal columns may be different. In an example, the stimulation parameter generator 950 may automatically determine the target current source poles based on the neural structures identified using the features extracted by the feature extractor 822.

The stimulation parameter generator 950 may then map the electric field (corresponding to the target current source pole configuration) to the electrode polarity 952 and the current fractionalization 954 for the electrodes on the stimulation lead. In an example, the mapping involves application of a transfer matrix to the target electrical field. The transfer matrix may be used to compute relative strengths of a plurality of constituent current sources needed to match the target electrical field at the spatial observation points when a specified optimization criterion is satisfied. In another example, the mapping involves a regression fitting of the target electrical field, such as by using a linear or nonlinear regression model. In an example, the mapping may involve a least-square fitting of the target electrical field. Examples of electric field estimation and stimulation parameter determination based on analytical and/or numerical model may include, but are not limited to, those described in U.S. Pat. No. 8,412,345, entitled “SYSTEM AND METHOD FOR MAPPING ARBITRARY ELECTRIC FIELDS TO PRE-EXISTING LEAD ELECTRODES”, assigned to Boston Scientific Neuromodulation Corporation, which are incorporated herein by reference in its entirety.

The amplitude threshold 956 represents a minimal stimulation amplitude required to activate one or more neural elements. In some example, based on the amplitude threshold 956, a boost or scaling factor may be determined for globally adjusting the magnitude of the total current supplied to the electrodes to maintain a perceived intensity level of the electrical stimulation. The stimulation pulse parameters 958 includes pulse amplitude, pulse width, pulse frequency, pulse waveform, duty cycle, or pacing duration, among other parameters.

FIGS. 10A-10B illustrate, by way of example and not limitation, views of a numerical CSC model. The CSC model is a volumetric model created using finite-element method (FEM), hereinafter referred to as an FEM spinal cord model. An example of the FEM spinal cord model is described in Lee et al., “Predicted effects of pulse width programming in spinal cord stimulation: a mathematical modeling study”, Medical and Biological Engineering and Computing, (2011) 49:765-774. Size, shape, appearance, volume, morphology, and positions of various neural elements may be modeled based on data or empirical knowledge generalized from a patient population. The CSC model include models of various spinal structures, and a cylindrical multi-contact lead model. FIG. 10A is a cross-sectional view 1010 of the FEM spinal cord model. By way of example and not limitation, the model includes spinal cord gray matter 1011, white matter 1012, cerebrospinal fluid (CSF) 1013, dura 1014, epidural space 1015, vertebral bone 1016, and surrounding environment 1017. Size and shape of various neural structures are each approximated based on population-based spinal cord data. In addition to the spinal structures, an electrode model 1018 on a lead model 1022 (as illustrated in FIG. 10B) is also illustrated. FIG. 10B illustrates a three-dimensional (3D) mesh representation 1020 of a portion of the FEM spinal cord model, including a spinal structure model 1021 and a lead model 1022. The volume of the spinal cord may be meshed over nodes with tetrahedron shaped elements, with a high-density mesh in the region close to where electrodes are located. The lead model 1022 includes an array of electrodes 1223, such as ring electrodes or directional electrodes. The lead model 1022 may be positioned dorsally, atop the dura and aligned with the midline of the spinal cord, as illustrated in FIG. 10B. The lead model 1220 may be modeled based on the lead 406, which includes eight cylindrical contacts separated by insulating polymer. In another example, the lead model includes sixteen cylindrical contacts separated by insulating polymer. Also shown in FIG. 10 includes target current source poles 1025 for generating the electric field for spinal cord stimulation. The target current source poles 1025 is in a multipolar configuration, which includes a center cathode and two anodes distributed along the longitudinal direction of the lead model 1022.

The CSC model may be displayed on a screen of the presentation device 811, and may be interactively manipulated (e.g., drag, rotate, zoom in or out, among other object controls) by a user via the user input device 812.

FIGS. 11A-11C illustrates, by way of example and not limitation, patient images and morphological features extracted therefrom for used to generate a patient-specific CSC model. As illustrated in FIG. 11A, the patient dataset is represented by a lumbar spinal cord magnetic resonance (MR) image 1110. To enhance the presentation of microstructures of the neural elements, more advanced diagnostic imaging techniques, such as diffusion-weighted imaging based on MRI, may be used. Heavy diffusion weighting may help suppress unwanted tissue structures such as fat, muscles, tendons, or blood vessels. The MR image 1110 may be displayed on a screen of the presentation device 811. The data processor 820 may process the MR image 1110 to recognize one or more neural structures including, for example, nerve roots 1112 and dorsal root ganglion (DRG) 1114, among others. The feature extractor 822 may extract one or more morphological features from, and/or perform measurements of, the recognized neural structures. FIG. 11B illustrates a schematic drawing 1120 of the MR image 1110, and various morphological features or measurements taken from the MR image 1110. Examples of the features, as illustrated in FIG. 11B, may include angulation, length, and width of a DRG, a trajectory or angulation of a dorsal root, thickness measurement of the dorsal cerebral spinal fluid, among others. FIG. 11C illustrates another spinal cord MR image 1130. The data processor 820 may process the image 1130 to recognize various spinal structures, such as a dorsal horn 1310 and bony anatomy (e.g., vertebrae). The feature extractor 822 may measure dorsal cerebrospinal fluid (CSF) thickness.

The spinal structure features or measurements taken from the MR images 1110 and 1130 may be used to modify the CSC model to generate a patient-specific model. In an example, locations and morphological measurements of the nerve roots 1112 and the 1114 may be used to control stimulation intensity (e.g., current amplitude). The intensity of stimulation current may be kept constant as the central point of stimulation (CPS) is moved along the trajectory of a recognized dorsal root (e.g., the nerve root 1112). The CPS represents a center of stimulation field for the electrode combination. Using a patient-specific CSC model, electrode polarity and current fractionalization may be determined, such that stimulation amplitude may be increased as the CPS approaches a recognized DRG or nerve root, and decreased as the CPS moves away from the recognized DRG or nerve root. In another example, the CSF thickness 1310 may be evaluated at various locations along the spinal cord on a vertebral level to determine physiological or pathological spatial variation of CSF thickness (e.g., due to degenerative disc disease). Using a patient-specific CSC model modified from the CSC model 919, electrode polarity and current fractionalization may be determined such that the stimulation intensity may be varied at least according to the CSF thickness. For example, through perception-based programming, a lower stimulation intensity may be applied to spinal locations with a shallow CSF (e.g., where the CSF thickness falls below a threshold value). Through sub-perception based programming, a higher stimulation intensity may be applied to the spinal cord location with a thick CSF (e.g., where the CSF thickness exceeds a threshold value). CSF is related to electrode distance of the stimulating electrodes (on the dura) to the dorsal columns. The thicker the CSF, the further the electrodes from the target tissue and, thus, requiring a higher stimulation intensity for thicker CSF values than smaller CSF values.

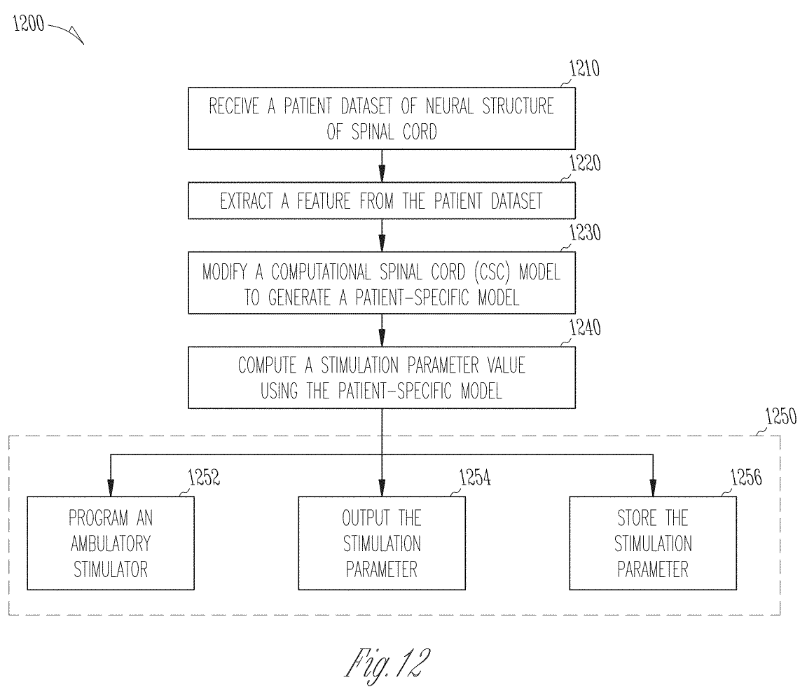

FIG. 12 illustrates, by way of example and not limitation, a flow chart of a method 1200 for controlling delivery of electrostimulation to neural targets, such as a spinal cord, via a medical system such as the neurostimulation system 100. The method 1200 may be implemented in an external device, such as one of the external devices 102 or 505, or the external programming device 802. In an example, at least a portion of the method 1200 may be implemented in, and executed by, the CP 630 and/or the RC 632. By executing the method 1200, the external device may program an implantable electrostimulator to deliver spinal cord stimulation, such as the stimulation device 104, one of the IPGs 404, 504, or 604, or the implantable stimulator 704. Although spinal cord stimulation is discussed herein, the method 1200 may be modified to provide stimulation of other neural targets, such as in the application of DBS, PNS, VNS, or other types of neurostimulation.

The method 1200 commences at 1210, where a patient spinal cord dataset is received, such as from a user via a user input device 812, or from a patient database such as an electronic medical record system. The dataset contains information about shape, appearance, size, morphology, or other geometric characteristics of one or more neural structures of the patient spinal cord. The patient dataset may take a format of an image, a point cloud, a parametric model, or other data formats. In an example, the patient dataset is a medical image generated with fluoroscopy, MRI, CT scan, CAT scan, PET scan, ultrasonography, or nuclear functional imaging, among other imaging modalities. The medial image has a specified data format. In some examples, metadata associated with the image may also be included in the patient dataset.

At 1220, a feature may be extracted from the patient dataset, such as by using the data processor 820. The feature may include a morphological feature representing position, orientation, thickness, spatial distance, angulation, among other geometric metrics of one or more neural structures of the patient spinal cord. By way of example and not limitation, the neural structures of the spinal cord may include gray matter, white matter, a dorsal root, a DRG, or CSF, among others. The neural structures may be recognized or classified into one of a number of types of neural elements using a machine-learning method, such as via the data processor 820. The recognition of neural structures may be used to select target poles, as previously discussed with reference to FIG. 8. In some examples, the extracted feature may additionally include features taken from other spinal structures such as the bony anatomy of the spinal cord, including inter-disc space, vertebral size, location and size of intervertebral foramen, etc. In some examples, when only bony anatomy is available, and neural structures are not available, the extracted features from the bony anatomy may be used to generalize features of neural structures, such as locations of one or more neural elements. For example, if patient dataset is a fluoroscopy image, then only bony anatomy extracted features (e.g., pedicle location or foramen location) are available and no neural tissue features can be extracted from the fluoroscopy image. Neural element features such as root trajectories can be predicted Based on pedicle location, or DRG location may be estimated from the foramen location.

In various examples, the type of the extracted feature may be dependent on the CSC model. For example, if the CSC model determines stimulation parameters using input parameters such as the position, orientation, thickness, spatial distance, angulation, or other geometric metrics of neural elements, then one or more of these features may be extracted to modify the CSC model.

At 1230, a patient-specific model may be generated via the programming control circuit 816. The patient-specific model may be a modification of a generic computational spinal cord (CSC) model. The CSC model characterizes anatomical and physical properties of neural structures of patient spinal cord, as well as various parameters and building blocks for delivery of spinal cord stimulation. The anatomical properties may include size, shape, or other geometric metrics of the spinal cord and various neural structures therein. The physical properties may include spinal cord mechanical, electrical, optical, or chemical properties. The CSC model may be in a form of a parametric model, a statistical model, a shape-based model, or a volumetric model. The CSC model may be a numerical model (e.g., an FEM spinal cord model), or an analytical model. In an example, the CSC model may an approximation of a spinal cord FEM model, where at least a portion of the modeled region is represented by an analytical model. The CSC model can be a generic spinal cord model created using spinal cord data generalized from a patient population. In addition to models of various spinal structures, the CSC model may additionally include a model of a stimulation lead with an electrode array. Information of electrode positions and/or orientation relative to the patient spinal cord contained in lead model may be used to determine stimulation parameters such as current fractionalization and polarity. An example of a spinal cord FEM model, along with a model of a multi-contact lead, is illustrated in FIGS. 10A-B, as discussed above.

The spinal structures the CSC model, such as the spinal structure model 920, may be updated using the features extracted from the patient dataset. Examples of the patient-specific features used for model modification may include measurements of dorsal cerebral spinal fluid thickness, location and/or morphology of a dorsal root relative to the electrodes of the stimulation lead, location and/or morphology of a dorsal root ganglion (e.g., an angulation, a length, or a width of dorsal root ganglia), or location and/or morphology of a dorsal horn relative to the electrodes of the stimulation lead, among others, as discussed above with reference to FIGS. 11A-11C.

At 1240, one or more stimulation parameters may be determined using the patient-specific model. The CSC model may accept relative electrode positions and a representation of a target electric field as model input, and maps the target electric field to the electrodes, thereby yielding polarities and the current fractionalization for the electrodes on the stimulation lead, among other stimulation parameters. In some examples, the CSC model may compute a threshold stimulation amplitude representing a minimal stimulation amplitude required to activate one or more neural elements. In some example, based on the amplitude threshold, a boost or scaling factor may be determined for globally adjusting the magnitude of the total current supplied to the electrodes to maintain a perceived intensity level of the electrical stimulation.

In an example, the target electric field may be estimated based on target current source pole configuration, such as by using the electric field estimator 940. The target current source poles are imaginary configuration of electrode locations with respective polarities. Target current source pole configuration refer to information about polarities (i.e., anodes or cathodes), locations, and current strength of target current source poles. Target current source poles, arranged in the identified configuration, may generate an electric field for stimulating various neural elements of the patient spinal cord.

Estimation of target electric field from the identified target current source pole configuration may be affected by anatomical and physical properties of neural elements of the patient spinal cord. The patient-specific model created at 1230, with patient-specific morphological features of neural structures, may be used to estimate a patient-specific target electric field. The electric field may then be mapped to electrode polarities and current fractionalization for the electrodes on the stimulation lead, such as via the stimulation parameter generator 950. In an example, the mapping involves application of a transfer matrix to the target electrical field. The transfer matrix may be used to compute relative strengths of a plurality of constituent current sources needed to match the target electrical field at the spatial observation points when a specified optimization criterion is satisfied. In another example, the mapping involves a regression fitting of the target electrical field, such as by using a linear or nonlinear regression model. In an example, the mapping may involve a least-square fitting of the target electrical field.

The stimulation parameter, such as one or more of the electrode polarities and current fractionalization for the electrodes on the stimulation lead, threshold stimulation amplitude or boost or scaling factors, or estimated electric field, among other parameters, may be output to a user or a process at 1250. In an example, at 1252, the stimulation parameter may be transmitted to an ambulatory device via a communication link such as the wireless communication link 640, to program the ambulatory device, such as the implantable stimulator 704. The ambulatory device may deliver electrostimulation (e.g., spinal cord stimulation) according to the programmed stimulation parameter value. The programming of the ambulatory device may be carried out automatically or triggered by a user command or a specific event. In another example, at 1254, the stimulation parameter may be presented to a user (e.g., a clinician or a patient), such as displayed on a display screen of the presentation device 811. The user may adjust the stimulation parameter through the user input device 812. In yet another example, at 1256, the stimulation parameter may be stored in a storage device, such as the external storage device 818 of the external programming device 802.

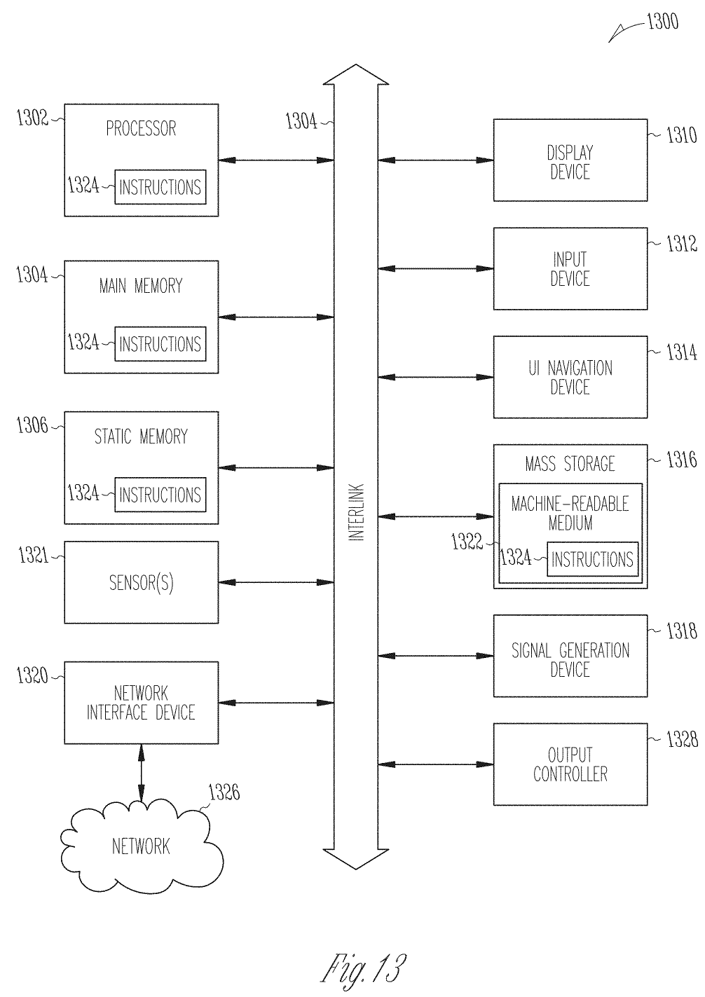

FIG. 13 illustrates generally a block diagram of an example machine 1300 upon which any one or more of the techniques (e.g., methodologies) discussed herein may perform. Portions of this description may apply to the computing framework of various portions of the LCP device, the IMD, or the external programmer.

In alternative embodiments, the machine 1300 may operate as a standalone device or may be connected (e.g., networked) to other machines. In a networked deployment, the machine 1300 may operate in the capacity of a server machine, a client machine, or both in server-client network environments. In an example, the machine 1300 may act as a peer machine in peer-to-peer (P2P) (or other distributed) network environment. The machine 1300 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1300 may include a hardware processor 1302 (e.g., a central processing unit (CPU), a graphics processing unit (GPU), a hardware processor core, or any combination thereof), a main memory 1304 and a static memory 1306, some or all of which may communicate with each other via an interlink (e.g., bus) 1308. The machine 1300 may further include a display unit 1310 (e.g., a raster display, vector display, holographic display, etc.), an alphanumeric input device 1312 (e.g., a keyboard), and a user interface (UI) navigation device 1314 (e.g., a mouse). In an example, the display unit 1310, input device 1312 and UI navigation device 1314 may be a touch screen display. The machine 1300 may additionally include a storage device (e.g., drive unit) 1316, a signal generation device 1318 (e.g., a speaker), a network interface device 1320, and one or more sensors 1321, such as a global positioning system (GPS) sensor, compass, accelerometer, or other sensor. The machine 1300 may include an output controller 1328, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1316 may include a machine readable medium 1322 on which is stored one or more sets of data structures or instructions 1324 (e.g., software) embodying or utilized by any one or more of the techniques or functions described herein. The instructions 1324 may also reside, completely or at least partially, within the main memory 1304, within static memory 1306, or within the hardware processor 1302 during execution thereof by the machine 1300. In an example, one or any combination of the hardware processor 1302, the main memory 1304, the static memory 1306, or the storage device 1316 may constitute machine readable media.

While the machine readable medium 1322 is illustrated as a single medium, the term “machine readable medium” may include a single medium or multiple media (e.g., a centralized or distributed database, and/or associated caches and servers) configured to store the one or more instructions 1324.

The term “machine readable medium” may include any medium that is capable of storing, encoding, or carrying instructions for execution by the machine 1300 and that cause the machine 1300 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1324 may further be transmitted or received over a communications network 1326 using a transmission medium via the network interface device 1320 utilizing any one of a number of transfer protocols (e.g., frame relay, interne protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1320 may include one or more physical jacks (e.g., Ethernet, coaxial, or phone jacks) or one or more antennas to connect to the communications network 1326. In an example, the network interface device 1320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1300, and includes digital or analog communications signals or other intangible medium to facilitate communication of such software.

Various embodiments are illustrated in the figures above. One or more features from one or more of these embodiments may be combined to form other embodiments.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for controlling delivery of electrostimulation to a patient spinal cord, the system comprising:
a data processor configured to receive a patient dataset representing a neural structure of at least a portion of the patient spinal cord, and to extract a feature from the received patient dataset; and
a programming control circuit configured to receive a computational spinal cord (CSC) model characterizing spinal cord anatomical and physical properties, the programming control circuit configured to:
modify the CSC model to generate a patient-specific model based on the extracted feature; and
compute a stimulation parameter value using the patient-specific model.

2. The system of claim 1, wherein the CSC model is a finite-element method (FEM) model.

3. The system of claim 1, wherein the CSC model characterizes the spinal cord physical properties including one or more of spinal cord mechanical, electrical, optical, or chemical properties.

4. The system of claim 1, wherein the CSC model comprises a lead model including information about position and orientation of electrodes on a stimulation lead relative to the patient spinal cord , wherein the programming control circuit is configured to compute, based at least in part on the lead model, a stimulation parameter value including polarities and current fractionalization of the electrodes on the stimulation lead.

5. The system of claim 4, wherein the programming control circuit is configured to use the patient-specific model to:
identify locations of target current source poles using the extracted feature;
determine a target electric field using the locations of the target current source poles for activating one or more neural elements; and
determine the polarities and the current fractionalization of the electrodes on the stimulation lead using the determined target electric field.

6. The system of claim 5, wherein the programming control circuit is configured to determine the polarities and the current fractionalization by applying a transfer matrix to the target electric field.

7. The system of claim 5, wherein the programming control circuit is configured to determine the polarities and the current fractionalization using a least-square fit of the target electric field.

8. The system of claim 5, wherein the programming control circuit is further configured to determine a threshold stimulation amplitude based on the target electric field, the threshold stimulation amplitude representing a minimal stimulation amplitude required to activate one or more neural elements.

9. The system of claim 1, wherein the received patient dataset includes a medical image of at least the portion of the patient spinal cord.

10. The system of claim 1, wherein the received patient dataset characterizes morphology of the neural structure of at least a portion of the patient spinal cord, and the data processor is configured to:
recognize a neural element of the patient spinal cord from the received patient dataset; and
extract a geometric feature of the recognized neural element.

11. The system of claim 10, wherein the geometric feature includes at least one of:
a cerebrospinal fluid thickness measurement;
location or morphology of a dorsal root;
location of morphology of a dorsal horn; or
location or morphology of a dorsal root ganglion.

12. The system of claim 1, further comprising a posture sensor to detect a change in patient posture, wherein:
the data processor is configured to receive a first patient dataset before the detected posture change and a second dataset after the detected posture change, and to determine a change from a first feature extracted from the first patient dataset to a second feature extracted from the second patient dataset; and
the programming control circuit is configured to modify the CSC model to generate the patient-specific model based on the determined change from the first extracted feature to the second extracted feature.

13. The system of claim 1, further comprising an ambulatory electrostimulator configured to stimulate the patient spinal cord according to the computed stimulation parameter value.

14. The system of claim 13, further comprising an external device that includes the data processor and the programming control circuit, the external device configured to be communicatively coupled to the ambulatory electrostimulator and to program the ambulatory electrostimulator with the computed stimulation parameter value.

15. A method for operating a medical system to control delivery of electrostimulation, the method comprising:
receiving a patient dataset representing a neural structure of at least a portion of the patient spinal cord;
extracting a feature from the received patient dataset via a data processor; and
modifying a computational spinal cord (CSC) model via a programming control circuit to generate a patient-specific model based on the extracted feature, the CSC model characterizing spinal cord anatomical and physical properties; and
computing, via the programming control circuit, a stimulation parameter value using the patient-specific model.

16. The method of claim 15, wherein:
the received CSC model further includes a lead model including information about position and orientation of electrodes on a stimulation lead relative to the patient spinal cord; and
the computed stimulation parameter value includes polarities and current fractionalization for the electrodes on the stimulation lead.

17. The method of claim 16, comprising:
identifying locations of target current source poles using the extracted feature;
determining a target electric field using locations of the target current source poles for activating one or more neural elements; and
determining the polarities and the current fractionalization of the electrodes on the stimulation lead using the determined target electric field.

18. The method of claim 15, wherein the received patient dataset includes a medical image of at least the portion of the patient spinal cord, and the extracted feature includes a geometric feature of a neural element extracted from the medical image.

19. The method of claim 15, wherein the geometric feature includes at least one of:
a cerebrospinal fluid thickness measurement;
location or morphology of a dorsal root;
location or morphology of a dorsal horn; or
location or morphology of a dorsal root ganglion.

20. The method of claim 15, further comprising:
via an external device, programming an ambulatory electrostimulator with the computed stimulation parameter value; and
via the ambulatory electrostimulator, delivering spinal cord stimulation according to the programmed stimulation parameter value.
